(19) United States
(12) Patent Application Publication
(10) Pub. No.: US 2022/0016377 A1
(43) Pub. Date: Jan. 20, 2022

(54) CPAP DEVICE

(71) Applicant: Murata Manufacturing Co., Ltd., Kyoto (JP)

(72) Inventors: Hiroki MAEDA, Kyoto (JP); Yoshihide AMAGAI, Kyoto (JP); Takahiro YAMAGUCHI, Kyoto (JP); Yoshitaka HANE, Kyoto (JP); Yoshio YAMANAKA, Kyoto (JP)

(21) Appl. No.: 17/490,105

(22) Filed: Sep. 30, 2021

Related U.S. Application Data
(63) Continuation of application No. PCT/JP2020/013725, filed on Mar. 26, 2020.

(30) Foreign Application Priority Data
Apr. 26, 2019 (JP) 2019-085816

(51) Int. Cl. A61M 16/08; A61M 16/16; A61M 16/00

(52) U.S. Cl. CPC A61M 16/0816; A61M 16/16; A61M 2039/1022; A61M 16/024; A61M 2205/121; A61M 16/0066

(57) ABSTRACT

The CPAP device includes a main unit and a second unit to be attached to or detached from the main unit. The main unit includes a first housing having a first lead-out part, an air blower for leading out air from the first lead-out part, and a first electrical connection part provided on the first housing. The second unit includes a second housing having a second lead-in part and a second lead-out part, a humidifier which humidifies air led therein from the second lead-in part, and a second electrical connection part to be connected to the first electrical connection part. The first lead-out part and the second lead-in part are configured to guide, with a connection between them, the first electrical connection part and the second electrical connection part to a position where they are connectable to each other.

### CROSS REFERENCE TO RELATED APPLICATION

This is a continuation of International Application No. PCT/JP2020/013725 filed on Mar. 26, 2020 which claims priority from Japanese Patent Application No. 2019-085816 filed on Apr. 26, 2019. The contents of these applications are incorporated herein by reference in their entireties.

### BACKGROUND OF THE DISCLOSURE

### Field of the Disclosure

The present disclosure relates to a continuous positive airway pressure (CPAP) device which feeds air suctioned into the device to the airway of a user.

### Description of the Related Art

In general, as a treatment for sleep-related disorders such as the obstructive sleep apnea syndrome (OSA), a continuous positive airway pressure (CPAP) device (hereinafter, CPAP device) which supplies a fluid to a user is used. The CPAP device is a device which has an air blowing device with a fan incorporated therein and supplies, from the air blowing device, a fluid (for example, air) to a mask attached to the mouth or nose of the user with a pressure higher than atmospheric pressure to open the airway (for example refer to Patent Document 1).
Patent Document 1: Japanese Unexamined Patent Application Publication No. 2016-34411

### BRIEF SUMMARY OF THE DISCLOSURE

Meanwhile, it has been known that humidifying air to be supplied to the user is useful for the user. Thus, a humidifying function is thought to be provided. On the other hand, the CPAP device is a device desired to be continuously used every day. Thus, for example, when the user sleeps away from home or the like, it is required to carry the CPAP device. Thus, for the CPAP device, the reduction in size and weight of the device is desired. However, providing the humidifier may decrease the portability of the device.

An object of the present disclosure is to provide a CPAP device with a humidifying function and high portability.

A CPAP device according to one aspect of the present disclosure is a CPAP device which feeds air led inside the device to an airway of a user. The CPAP device includes a first unit and a second unit to be attached to or detached from the first unit. The first unit includes a first housing having a first lead-in part and a first lead-out part, an air blower for leading out air led therein from the first lead-in part from the first lead-out part, a first electrical connection part provided on the first housing, and a first guide part. The second unit includes a second housing having a second lead-in part and a second lead-out part, a humidifier which humidifies air led therein from the second lead-in part, a second electrical connection part to be electrically connected to the first electrical connection part, and a second guide part. The first guide part and the second guide part are configured to guide, with a connection between the first lead-out part and the second lead-in part, the first electrical connection part and the second electrical connection part to a position where the first electrical connection part and the second electrical connection part are electrically connectable to each other.

According to this configuration, a CPAP device with a humidifying function and high portability can be provided.

According to one aspect of the present disclosure, a CPAP device with a humidifying function and high portability can be provided.

### BRIEF DESCRIPTION OF THE SEVERAL VIEWS OF THE DRAWINGS

FIG. 1 is a perspective view depicting a main unit and a base unit of a CPAP device in a first embodiment.

FIG. 2 is a perspective view of the CPAP device in the first embodiment viewed from another angle.

FIG. 3 is a perspective view depicting a state in which the base unit is attached to the main unit in the first embodiment.

FIG. 4A and FIG. 4B are partial sectional views each depicting a connecting portion between the main unit and the base unit in the first embodiment.

FIG. 5A is a schematic view depicting a first use state of the CPAP device in each embodiment and FIG. 5B is a schematic view depicting a second use state of the CPAP device.

FIG. 6 is a descriptive view depicting the configuration of functional blocks of the CPAP device in the first use state in each embodiment.

FIG. 7 is a descriptive view depicting the configuration of functional blocks of the CPAP device in the second use state in each embodiment.

FIG. 8 is a descriptive diagram depicting the functional blocks of the CPAP device in each embodiment.

FIG. 9 is a descriptive diagram depicting the outline of a CPAP device of a modification in the first embodiment.

FIG. 10 is a descriptive diagram depicting the outline of a CPAP device of a modification in the first embodiment.

FIG. 11 is a descriptive diagram depicting the outline of a CPAP device of a modification in the first embodiment.

FIG. 12 is a descriptive diagram depicting the outline of a CPAP device of a modification in the first embodiment.

FIG. 13 is a descriptive diagram depicting the outline of a CPAP device of a modification in the first embodiment.

FIG. 14 is a descriptive diagram depicting the outline of a CPAP device of a modification in the first embodiment.

FIG. 15A and FIG. 15B are descriptive diagrams each depicting a modification of a base part in the first embodiment.

FIG. 16 is a descriptive diagram depicting the outline of a CPAP device of a modification in the first embodiment.

FIG. 17 is a descriptive diagram depicting the outline of a CPAP device of a modification in the first embodiment.

FIG. 18 is a perspective view depicting a main unit and a base unit of a CPAP device of a modification in the first embodiment.

FIG. 19 is a perspective view depicting a main unit and a base unit of a CPAP device in a second embodiment.

FIG. 20 is a perspective view of the CPAP device in the second embodiment viewed from another angle.

FIG. 21 is a diagram describing a second electrical connection part movable in the second embodiment.

### DETAILED DESCRIPTION OF THE DISCLOSURE

In the following, a first embodiment is described.

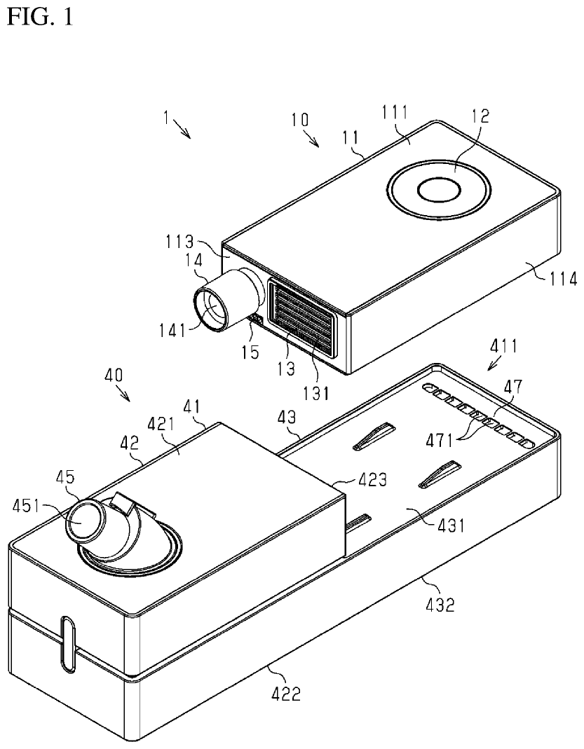

As depicted in FIG. 1 to FIG. 8, a CPAP device 1 includes a main unit 10 as a first unit and a base unit 40 as a second unit. The main unit 10 includes an air blower 22 as a main component. The base unit 40 includes a humidifier 51 as a main component.

The base unit 40 is attachable to and detachable from the main unit 10.

In the present embodiment, the CPAP device 1 is configured to be usable in a first use state and a second use state. The first use state is a state in which the base unit 40 is attached to the main unit 10, and the second use state is a state in which the base unit 40 is not attached to the main unit 10.

In the first use state, the main unit 10 and the base unit 40 are used. In the second use state, only the main unit 10 is used, and the base unit 40 is not used.

In the CPAP device 1 of the present embodiment, the CPAP device 1 is configured of a plurality of units and is configured so that, with the plurality of these units configured to be mutually attachable and detachable, enhanced convenience is achieved not only at home or the like but also at sleep away from home or the like. At home, with the base unit 40 attached to the main unit 10, the CPAP device 1 can be used in the above-described first use state. In this first use state, with the base unit 40 attached to the main unit 10, humidified air can be supplied to a user as the CPAP device 1 including a humidifier 51.

And, at sleep away from home or the like, the CPAP device 1 can be used in the second use state in which the base unit 40 is not attached to the main unit 10. In this second use state, since the CPAP device 1 is only with the main unit 10, it is only required to carry only the main unit 10 and high portability is obtained. Note that the CPAP device 1 can also be used at home in the second use state.

As depicted in FIG. 1 to FIG. 3, the main unit 10 has a flat outer shape in a substantially rectangular-parallelepiped shape, and its hull is configured of a first housing 11.

The first housing 11 has an upper surface 111 and a lower surface 112 oriented to be opposed to each other in a thickness direction and four side surfaces 113, 114, 115, and 116 connecting the upper surface 111 and the lower surface 112.

The upper surface 111 is an operation surface provided with an operation part 12. The operation part 12 includes an annular member and a plurality of switches to be turned ON and OFF by that member. Setting values can be changed by the switches turned ON and OFF.

The lower surface 112 is a mount surface to be mounted on the base unit 40 in the first use state and mounted on a floor surface, a table, or the like in the second use state. The side surface 113 is a connection surface to be connected to the base unit 40 in the first use state, and is hereinafter referred to as a first connection surface 113. Note that the side surface 113 is orthogonal to the upper surface 111 and the lower surface 112.

The first connection surface 113 of the first housing 11 is provided with a first lead-in part 13 which leads air outside the first housing 11 into the first housing 11 and a first lead-out part 14 which leads out air from the inside to the outside of the first housing 11. The first lead-out part 14 is one example of a first guide part and a convex part. The first lead-in part 13 has a plurality of through holes. With the plurality of these through holes, the first housing 11 has a first lead-in port 131. Also, the first connection surface 113 of the first housing 11 is provided with a first electrical connection part 15 for electrically connecting the main unit 10 and the base unit 40. In the present embodiment, the first electrical connection part 15 electrically connects the main unit 10 and the base unit 40. In the present embodiment, the first electrical connection part 15 is a connector having a terminal to be attached to and detached from a second electrical connection part 46 of the base unit 40.

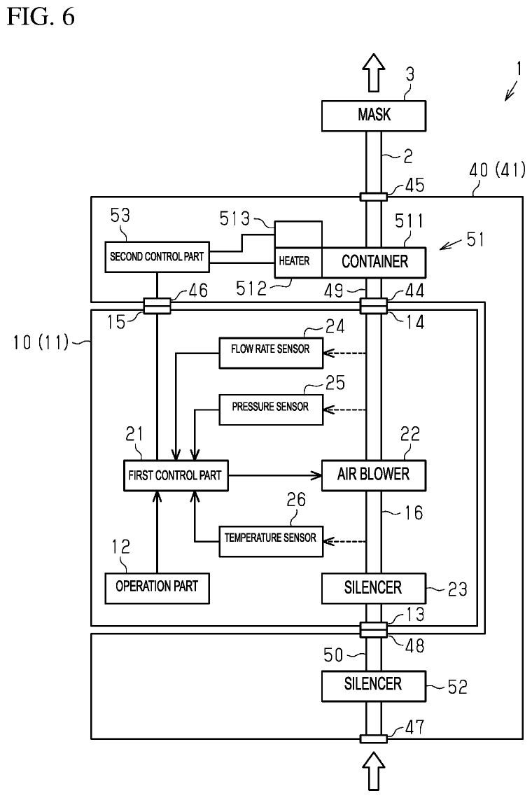

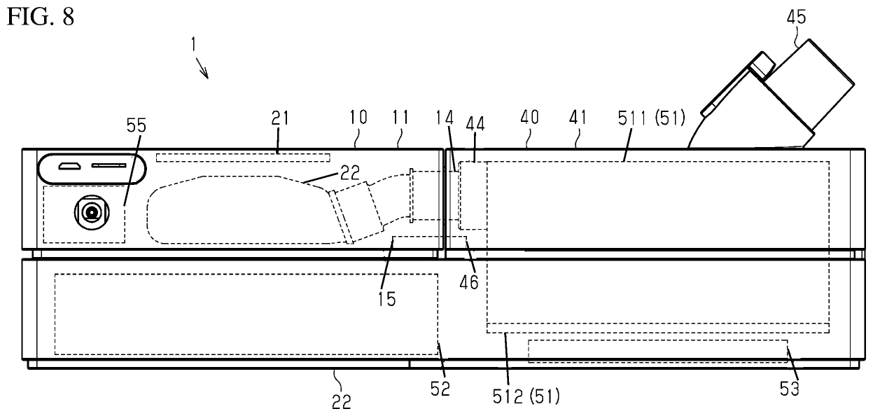

As depicted in FIG. 1 to FIG. 3, the base unit 40 includes a second housing 41. The second housing 41 has a humidifying part 42 provided with the humidifier 51 depicted in FIG. 6 and FIG. 8 and a mount part 43 where the main unit 10 is mounted.

The humidifying part 42 is formed to be thick, compared with the mount part 43. A lower surface 422 of the humidifying part 42 and a lower surface 432 of the mount part 43 are contiguous as being flush with each other. Therefore, an upper surface 421 of the humidifying part 42 is at a position higher than the upper surface 421 of the mount part 43. With the humidifying part 42 and the mount part 43, the second housing 41 has a slender outer shape in a substantially rectangular-parallelepiped shape partially provided with a deficient part 411 in a longitudinal direction. These lower surfaces 422 and 432 are mount surfaces to be mounted on a floor surface, a table, or the like in the first use state.

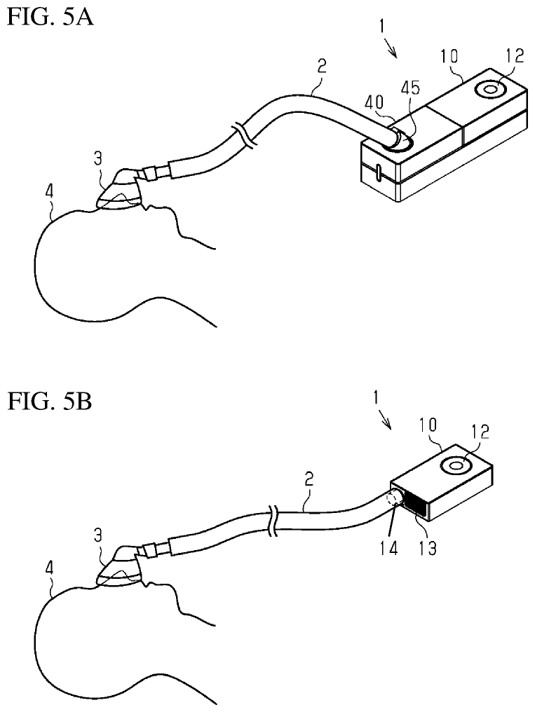

The upper surface 421 of the humidifying part 42 is provided with a second lead-out part 45. The second housing 41 has a second lead-out port 451 which leads out air from inside the second housing 41 through the second lead-out part 45. To this second lead-out part 45 (second lead-out port 451), an air tube 2 (refer to FIG. 5A and FIG. 5B) is connected in the first use state. An upper surface 431 of the mount part 43 is a surface where the main unit 10 is mounted. A difference in height between the upper surface 421 of the humidifying part 42 and the upper surface 431 of the mount part 43 corresponds to the thickness of the first housing 11 of the main unit 10.

In the humidifying part 42, a side surface facing the deficient part 411 is a second connection surface 423 to which the main unit 10 is connected.

This second connection surface 423 is provided with a second lead-in part 44 for leading air therein from the outside of the second housing 41. The second lead-in part 44 has a through hole and, with this through hole, the second housing 41 has a second lead-in port 441. This second connection surface 423 is provided with the second electrical connection part 46. With the base unit 40 attached to the main unit 10, the second electrical connection part 46 is electrically connected to the first electrical connection part 15 of the main unit 10. In the present embodiment, the second electrical connection part 46 is a connector having a terminal to be attached to and detached from the first electrical connection part 15.

The first electrical connection part 15 and the second electrical connection part 46 are to perform electrical attachment and detachment between the main unit 10 and the base unit 40. In the present embodiment, the first electrical connection part 15 and the second electrical connection part 46 are used for power supply to the base unit 40 and data transmission and reception using digital signals. Note that power supply and data transmission and reception can also be performed by separate connection parts. For example, noncontact power supply, short-distance wireless communication, and so forth can be used in combination.

The upper surface 431 of the mount part 43 of the second housing 41 described above is provided with a third lead-in part 47 which leads air therein from the outside of the second housing 41. The third lead-in part 47 has a plurality of through holes penetrating through the second housing 41 and, with the plurality of these through holes, the second housing 41 has a third lead-in port 471. The second connection surface 423 is provided with a third lead-out part 48 for leading out air from the second housing 41. The third lead-out part 48 has a through hole penetrating through the second housing 41 and, with this through hole, the second housing 41 has a third lead-out port 481.

With the main unit 10 arranged on the deficient part 411 of the base unit 40, the lower surface 112 of the first housing 11 and the upper surface 431 of the mount part 43 of the second housing 41 are opposed to each other, and the first connection surface 113 of the first housing 11 and the second connection surface 423 of the second housing 41 are opposed to each other. The first lead-out part 14 provided on the first connection surface 113 of the first housing 11 and the second lead-in part 44 provided on the second connection surface 423 of the second housing 41 are connected to each other. Also, the first lead-in part 13 provided on the first connection surface 113 of the first housing 11 and the third lead-out part 48 provided on the second connection surface 423 of the second housing 41 are connected to each other.

With this, the CPAP device 1 has a substantially rectangular-parallelepiped shape as a whole. Air led into the second housing 41 through the third lead-in part 47 of the second housing 41 is led into the first housing 11 via the third lead-out part 48 of the second housing 41 and the first lead-in part 13 of the first housing 11. Air inside the first housing 11 is led into the second housing 41 via the first lead-out part 14 of the first housing 11 and the second lead-in part 44 of the second housing 41. It is then led to the outside from the second lead-out part 45 of the second housing 41.

On the other hand, in a state in which the base unit 40 is not attached to the main unit 10, the first connection surface 113 of the first housing 11 is exposed toward the outside. Thus, the first lead-in part 13 and the first lead-out part 14 provided on the first connection surface 113 of the first housing 11 are in a state of being open toward the outside. With this, the main unit 10 leads air into the first housing 11 from the outside of the first housing 11 through the first lead-in part 13, and leads air inside the first housing 11 to the outside from the first lead-out part 14. That is, the main unit 10 functions singly as a CPAP device.

The connection between the first lead-out part 14 and the second lead-in part 44 is described in detail.

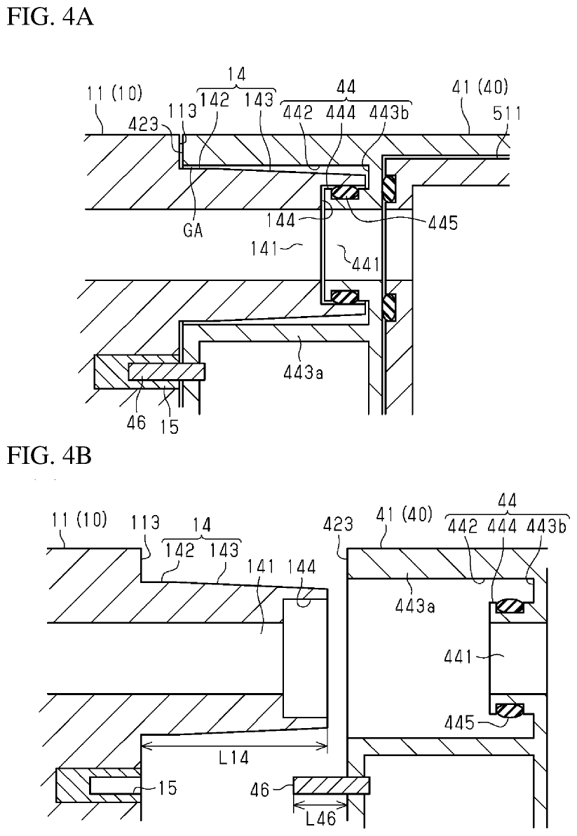

As depicted in FIG. 4A and FIG. 4B, the first lead-out part 14 protrudes from the first connection surface 113. The first lead-out part 14 includes a base part 142 and a connection cylinder part 143. The base part 142 is connected to the first connection surface 113. The connection cylinder part 143 extends from the base part 142 toward a direction opposite to the first connection surface 113. The base part 142 is formed in a cylindrical shape. The outer peripheral surface of the base part 142 is a cylindrical surface, and the outer shape dimension of the base part 142 is uniform toward a direction protruding from a first connection surface 113 side. The connection cylinder part 143 is formed in a tapered shape, with its outer diameter dimension decreased toward its tip. In other words, the first lead-out part 14 has the base part 142 having a cylindrical peripheral surface and the connection cylinder part 143 having a tapered peripheral surface.

The connection cylinder part 143 has, from its tip toward the base part 142, a connection concave part 144. The through hole penetrating through the base part 142, the connection cylinder part 143, and the connection concave part 144 makes the outside and the inside of the first housing 11 communicate. With this first lead-out part 14, the first housing 11 has a first lead-out port 141 which leads out air to the outside of the first housing 11.

The second lead-in part 44 has an accommodating part 442 and a connection convex part 444. The accommodating part 442 is one example of a second guide part and a concave part. The accommodating part 442 is recessed from the second connection surface 423 of the second housing 41 toward the inside of the second housing 41. As depicted in FIG. 4A, in the accommodating part 442, the first lead-out part 14 of the first housing 11 is accommodated.

The accommodating part 442 has an inner wall 443a and a wall part 443b. The inner wall 443a is in a cylindrical shape, and its inner diameter dimension is set slightly larger than the outer shape dimension of the base part 142 of the first lead-out part 14. That is, the CPAP device 1 has a gap (clearance) GA between the inner wall 443a (inner surface) of the accommodating part 442 and the peripheral surface of the base part 142 of the first lead-out part 14. The size of the gap GA is a distance between the base part 142 of the first lead-out part 14 and the inner wall 443a of the accommodating part 442, and its distance is set in accordance with the first electrical connection part 15 and the second electrical connection part 46.

The wall part 443b of the accommodating part 442 is provided with the connection convex part 444. The connection convex part 444 protrudes toward a cavity end of the accommodating part 442. The connection convex part 444 is formed in a cylindrical shape. With this connection convex part 444, the second housing 41 has the second lead-in port 441 which leads air into the inside of the second housing 41.

The outer peripheral surface of the connection convex part 444 is provided with a sealing member 445. The sealing member 445 is accommodated in a groove part provided along the outer peripheral surface of the connection convex part 444. The sealing member 445 is in close contact with the inner peripheral surface of the connection concave part 144 of the first lead-out part 14 of the first housing 11. The sealing member 445 is, for example, an annular seal member. This sealing member 445 seals a portion between the connection convex part 444 and the connection concave part 144. That is, this sealing member 445 prevents the leakage of the air from the portion between the first lead-out part 14 and the second lead-in part 44.

The first housing 11 is provided with the first electrical connection part 15. In the present embodiment, the first electrical connection part 15 is a female connector, and is provided inside the first housing 11 so as not to protrude from the first connection surface 113.

The second housing 41 is provided with the second electrical connection part 46. The second electrical connection part 46 partially protrudes from the second connection surface 423 of the second housing 41. In the present embodiment, the second electrical connection part 46 is a male connector. This second electrical connection part 46 is inserted into the first electrical connection part 15. With the first electrical connection part 15 and the second electrical connection part 46, the main unit 10 and the base unit 40 are electrically connected.

As depicted in FIG. 4B, a length L14 of the first lead-out part 14 is a length from the first connection surface 113 of the first housing 11 to the tip of the first lead-out part 14. A length L46 of the second electrical connection part 46 is a length from the second connection surface 423 of the second housing 41 to the tip of the second electrical connection part 46. The length L14 of the first lead-out part 14 is longer than the length L46 of the second electrical connection part 46. Therefore, when the base unit 40 is attached to the main unit 10, the tip of the first lead-out part 14 is first inserted into the second lead-in part 44, and then the second electrical connection part 46 is inserted into the first electrical connection part 15.

The first lead-out part 14 has the base part 142 having a cylindrical peripheral surface and the connection cylinder part 143 having a tapered peripheral surface.

Therefore, when the first lead-out part 14 is inserted into the second lead-in part 44, the connection cylinder part 143 having the tapered peripheral surface makes the first lead-out part 14 move toward the center of the second lead-in part 44. With this movement of the first lead-out part 14 with respect to the second lead-in part 44, the first lead-out part 14 is guided to a position where the connection convex part 444 of the second lead-in part 44 can be inserted into the connection concave part 144 of the first lead-out part 14. Similarly, with the movement of the first lead-out part 14 with respect to the second lead-in part 44, the first lead-out part 14 is guided to a position where the first electrical connection part 15 of the first housing 11 and the second electrical connection part 46 of the second housing 41 are electrically connectable to each other.

In the present embodiment, the outer diameter dimension of the connection cylinder part 143 on a base part 142 side is equal to the outer diameter dimension of the base part 142. The outer shape dimension of that base part 142 is smaller than the inner diameter dimension of the accommodating part 442 of the second housing 41. Therefore, as depicted in FIG. 4A, the base part 142 is inserted into the accommodating part 442. The gap (clearance) GA occurs between the outer peripheral surface of the base part 142 and the inner peripheral surface of the accommodating part 442. With this gap GA provided, the first housing 11 and the second housing 41 can be relatively moved along the first connection surface 113 and the second connection surface 423. This movement allows further alignment between the first electrical connection part 15 and the second electrical connection part 46.

FIG. 5A and FIG. 5B schematically depict the use states of the CPAP device 1 of the present embodiment. FIG. 5A depicts the first use state, and FIG. 5B depicts the second use state.

As depicted in FIG. 5A, in the first use state, the CPAP device 1 is used in a state in which the base unit 40 is attached to the main unit 10. In this case, a first end portion of the air tube 2 is connected to the second lead-out part 45 provided on the base unit 40, and a second end portion of the air tube 2 is connected to a mask 3. The mask 3 is attached so as to, for example, cover the nose or mouth of a user 4.

In this first use state, with the air blower 22 provided on the main unit 10 driven, air is suctioned from the third lead-in part 47 provided on the base unit 40 into the inside of the CPAP device 1, and air inside the CPAP device 1 is exhausted to the outside from the second lead-out part 45 provided on the base unit 40. This causes air to be fed to the airway of the user 4 via the air tube 2 and the mask 3.

As depicted in FIG. 5B, in the second use state, in the CPAP device 1, only the main unit 10 with the base unit 40 not attached thereto is used. In this case, the first end portion of the air tube 2 is connected to the first lead-out part 14 provided on the main unit 10, and the second end portion of the air tube 2 is connected to the mask 3. The mask 3 is attached so as to, for example, cover the nose or mouth of the user 4.

In this second use state, with the air blower 22 provided on the main unit 10 driven, air is suctioned from the first lead-in part 13 provided on the main unit 10 into the inside of the CPAP device 1, and air inside the CPAP device 1 is exhausted to the outside from the first lead-out part 14 provided on the main unit 10. This causes air to be fed to the airway of the user 4 via the air tube 2 and the mask 3.

Next, the configuration of functional blocks of the CPAP device 1 of the present embodiment is described.

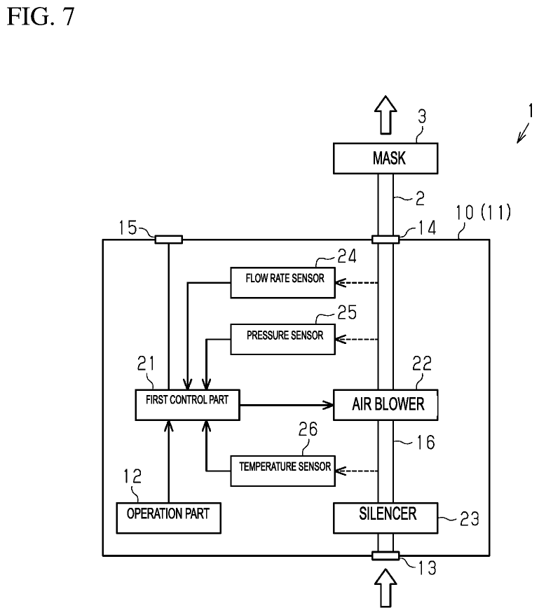

FIG. 6 depicts the configuration of functional blocks of the CPAP device 1 of the present embodiment in the first use state, and FIG. 7 depicts the configuration of functional blocks of the CPAP device 1 in the second use state.

As depicted in FIG. 6 and FIG. 7, the main unit 10 of the CPAP device 1 includes a first control part 21, the operation part 12, the air blower 22, a silencer 23, a flow rate sensor 24, a pressure sensor 25, and a temperature sensor 26. The base unit 40 includes the humidifier 51, a silencer 52, and a second control part 53.

The main unit 10 includes a first flow path 16 between the first lead-in part 13 and the first lead-out part 14. The first flow path 16 is provided with the air blower 22. The air blower 22 is configured of, for example, a centrifugal fan. The air blower 22 is installed in an air blower chamber provided in the first housing 11. The air blower chamber is part of the first flow path 16 and, with this air blower chamber, the air blower 22 is arranged on the first flow path 16.

In the first flow path 16, the silencer 23 is provided between the first lead-in part 13 and the air blower 22. The silencer 23 suppresses the leakage of the noise (such as operation sound of a driving motor and wind noise) occurring at the air blower 22 to the outside of the first housing 11.

The first flow path 16 is provided with the pressure sensor 25, the flow rate sensor 24, and the temperature sensor 26. The pressure sensor 25 is a sensor for detecting the pressure of air sent by the air blower 22. The flow rate sensor 24 is a sensor for detecting a flow rate of air between the main unit 10 and the air tube 2. The temperature sensor 26 is a sensor for detecting temperature of air led in from the outside of the main unit 10.

The first control part 21 includes, as main components, an arithmetic processing part (central processing unit: CPU) which executes a program, a storage part (read only memory: ROM/random access memory: RAM), a communicating part, a driving part which causes the air blower 22 to operate, and so forth. In the storage part, a ROM which stores data in a non-volatile manner and a RAM which stores data in a volatile manner are included. The data stored in the storage part includes data generated by execution of a program by the arithmetic processing part, data inputted via the operation part 12, data measured by various sensors described above, data to be transmitted to and received from the outside by the communicating part, and so forth.

The process in the arithmetic processing part is implemented by each piece of hardware and software executed by the arithmetic processing part. This software is stored in advance in the storage part. Acceptance of operation on the operation part 12, control of a driving motor which drives the air blower 22, external communication, and so forth are also implemented by the software.

In the present embodiment, based on the flow rate value and the pressure value detected by the flow rate sensor 24 and the pressure sensor 25, the first control part 21 performs control such as feedback control or feed-forward control to increase or decrease the number of revolution of the air blower 22, thereby controlling the amount of sending air and so forth. For example, based on the measurement values by the flow rate sensor 24 and the pressure sensor 25, the first control part 21 determines an exhalation state of the user and controls the pressure value of air to be supplied to the user so that the pressure value synchronizes with that exhalation state. Also, the first control part 21 transmits the temperature value detected by the temperature sensor 26 to the outside.

The second housing 41 of the base unit 40 includes a second flow path 49 between the second lead-in part 44 and the second lead-out part 45 and a third flow path 50 between the third lead-in part 47 and the third lead-out part 48. The second flow path 49 is provided with the humidifier 51. In the first use state, the humidifier 51 adds humidity to air flowing through the second flow path 49. With this, the CPAP device 1 supplies appropriate moisture to air to be sent toward the airway of the user. In the present embodiment, the humidifier 51 is a thermal humidifying device.

As depicted in FIG. 6, the humidifier 51 includes a container 511, a heater 512, and a temperature sensor 513. The container 511 is configured to be attachable to and detachable from the second housing 41. The container 511 stores water. Also, the container 511 has a lead-in tube which communicates with the second lead-in part 44 and, via that lead-in tube, air is supplied into the container 511. The container 511 has a heater pad for the heater 512. The heater 512 heats water in the container 511. The temperature sensor 513 detects temperature of the heater 512.

The second control part 53 includes, as with the above-described first control part 21, an arithmetic processing part (CPU) which executes a program, a storage part (ROM/RAM), a communicating part, a driving part which causes the humidifier 51 to operate, and so forth as main components. In the storage part, a ROM which stores data in a non-volatile manner and a RAM which stores data in a volatile manner are included. The data stored in the storage part includes data generated by execution of a program by the arithmetic processing part, heater temperature measured by the temperature sensor 513, data to be transmitted to and received from the first control part 21 by the communicating part in the first use state, and so forth. The data received from the first control part 21 includes an air temperature value measured by the temperature sensor 26 of the main unit 10. Note that as data received from the first control part 21, data inputted via the operation part 12 may be included.

The process in the arithmetic processing part is implemented by each piece of hardware and software executed by the arithmetic processing part. This software is stored in advance in the storage part. Control of the heater 512 of the humidifier 51, communication with the first control part 21, and so forth are also implemented by the software.

In the present embodiment, based on the air temperature received from the first control part 21, the second control part 53 sets a target temperature for heating water in the container 511. For example, the second control part 53 sets a target temperature by using, for example, a table or calculation expression stored in the storage part. Then, based on the heater temperature detected by the temperature sensor 513, the second control part 53 performs control such as feedback control or feed-forward control to drive the heater 512 so as to cause the heater temperature to become the target temperature. By that heater 512, the water temperature in the container 511 is adjusted. With this, a desired humidity is supplied to air passing through the inside of the container 511.

Note that the second control part 53 may stop the humidifier 51, that is, stop control of the heater 512, based on the air temperature received from the first control part 21.

The third flow path 50 is provided with the silencer 52. As depicted in FIG. 8, in the base unit 40, the silencer 52 is provided in the mount part 43 where the main unit 10 is mounted. This silencer 52 has a size slightly smaller than the size (outer shape) of the main unit 10. In the silencer 52, as its capacity is larger, the silent characteristics of the CPAP device 1 are enhanced.

As the silencer 52, it is configured of, for example, a resonant tube branched from the third flow path 50. The resonant tube is configured of a meandering flow path. The resonant tube is also called a Helmholtz resonator or resonator, having a function of attenuating noise in a predetermined frequency band. The resonant tube attenuates sounds in a frequency band determined by a natural frequency.

As depicted in FIG. 8, the main unit 10 includes a power supply part 55. To the power supply part 55, an AC adaptor not depicted is connected. The power supply part 55 generates operating power required for the main unit 10 and the base unit 40. The operating power is supplied to the second control part 53 of the base unit 40 and so forth via the first electrical connection part 15 of the main unit 10 and the second electrical connection part 46 of the base unit 40.

Next, the operation of the above-described CPAP device 1 is described.

The CPAP device 1 includes the main unit 10 and the base unit 40 to be attached to the main unit 10.

Regarding the CPAP device 1, at home, with the base unit 40 attached to the main unit 10, the CPAP device 1 can be used in the above-described first use state. In this first use state, with the base unit 40 attached to the main unit 10, humidified air can be supplied to the user as the CPAP device 1 including the humidifier 51. At sleep away from home or the like, the CPAP device 1 can be used in the second use state in which the base unit 40 is not attached to the main unit 10. In this second use state, since the CPAP device 1 is only with the main unit 10, it is only required to carry only the main unit 10 and high portability is obtained.

The first housing 11 of the main unit 10 has the first lead-in part 13, the first lead-out part 14, and the first electrical connection part 15. The first lead-out part 14 and the first electrical connection part 15 are provided on the first connection surface 113 of the first housing 11. The second housing 41 of the base unit 40 has the second lead-in part 44, the second lead-out part 45, and the second electrical connection part 46. The second lead-in part 44 and the second electrical connection part 46 are provided on the second connection surface 423 opposed to the first connection surface 113 of the first housing 11 in the first use state.

The first lead-out part 14 has the annular base part 142 and the connection cylinder part 143 extending from the base part 142. The connection cylinder part 143 is formed in a tapered shape, with its outer diameter dimension decreased toward its tip. In other words, the first lead-out part 14 has the base part 142 having a cylindrical peripheral surface and the connection cylinder part 143 having a tapered peripheral surface.

The first lead-out part 14 includes the base part 142 and the connection cylinder part 143. The base part 142 is connected to the first connection surface 113. The connection cylinder part 143 extends from the base part 142 toward a direction opposite to the first connection surface 113. The base part 142 is formed in a cylindrical shape. The outer peripheral surface of the base part 142 is a cylindrical surface, and the outer shape dimension of the base part 142 is uniform toward a direction protruding from a first connection surface 113 side. The connection cylinder part 143 is formed in a tapered shape, with its outer diameter dimension decreased toward its tip.

The second lead-in part 44 has the accommodating part 442 and the connection convex part 444. The accommodating part 442 is recessed from the second connection surface 423 of the second housing 41 toward the inside of the second housing 41. As depicted in FIG. 4A, in the accommodating part 442, the first lead-out part 14 of the first housing 11 is accommodated.

When the first lead-out part 14 is inserted into the second lead-in part 44, the connection cylinder part 143 having the tapered peripheral surface makes the first lead-out part 14 move toward the center of the second lead-in part 44. With this movement of the first lead-out part 14 with respect to the second lead-in part 44, the first lead-out part 14 is guided to a position where the connection convex part 444 of the second lead-in part 44 can be inserted into the connection concave part 144 of the first lead-out part 14. Therefore, the first lead-out part 14 and the second lead-in part 44 can be easily connected.

Also, with the movement of the first lead-out part 14 with respect to the second lead-in part 44, the first housing 11 and the second housing 41 are relatively guided to a position where the first electrical connection part 15 of the first housing 11 and the second electrical connection part 46 of the second housing 41 are electrically connectable to each other. That is, the first lead-out part 14 and the second lead-in part 44 function as a first guide part and a second guide part which guide the first electrical connection part 15 and the second electrical connection part 46 to a position so that they are electrically connectable to each other. This configuration allows the first electrical connection part 15 and the second electrical connection part 46 to be easily connected.

In the CPAP device 1 of the present embodiment, the outer diameter dimension of the connection cylinder part 143 on a base part 142 side is equal to the outer diameter dimension of the base part 142. The outer shape dimension of that base part 142 is smaller than the inner diameter dimension of the accommodating part 442 of the second housing 41. Therefore, as depicted in FIG. 4A, the base part 142 is inserted into the accommodating part 442. The gap (clearance) GA occurs between the outer peripheral surface of the base part 142 and the inner peripheral surface of the accommodating part 442. With this gap GA provided, the first housing 11 and the second housing 41 can be relatively moved along the first connection surface 113 and the second connection surface 423. This movement allows further alignment between the first electrical connection part 15 and the second electrical connection part 46.

The connection cylinder part 143 of the first lead-out part 14 has, from its tip toward the base part 142, the connection concave part 144. The through hole penetrating through the base part 142, the connection cylinder part 143, and the connection concave part 144 makes the outside and the inside of the first housing 11 communicate. With this first lead-out part 14, the first housing 11 has the first lead-out port 141 which leads out air to the outside of the first housing 11.

The connection convex part 444 of the second lead-in part 44 is provided on the wall part 443b of the accommodating part 442. The connection convex part 444 protrudes toward the cavity end of the accommodating part 442. The connection convex part 444 is formed in a cylindrical shape. With this connection convex part 444, the second housing 41 has the second lead-in port 441 which leads air into the inside of the second housing 41.

The outer peripheral surface of the connection convex part 444 is provided with the sealing member 445. The sealing member 445 is accommodated in the groove part provided along the outer peripheral surface of the connection convex part 444. The sealing member 445 is in close contact with the inner peripheral surface of the connection concave part 144 of the first lead-out part 14 of the first housing 11. The sealing member 445 is, for example, an annular seal member. This sealing member 445 seals a portion between the connection convex part 444 and the connection concave part 144. That is, this sealing member 445 prevents the leakage of the air from the portion between the first lead-out part 14 and the second lead-in part 44.

In the first use state in which the base unit 40 is attached to the main unit 10, the second control part 53 of the base unit 40 controls the humidifier 51 provided in the base unit 40. Therefore, the first control part 21 of the main unit 10 is only required to control the air blower 22 provided in the main unit 10 and accept operation of the operation part 12. Therefore, compared with the case in which the first control part 21 controls the entire CPAP device 1, the computation load of the first control part 21 and so forth can be suppressed low, and heating can be suppressed.

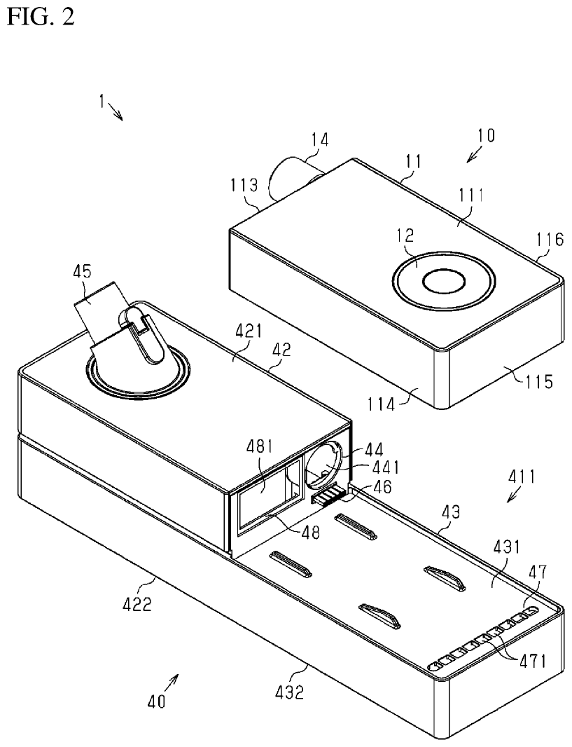

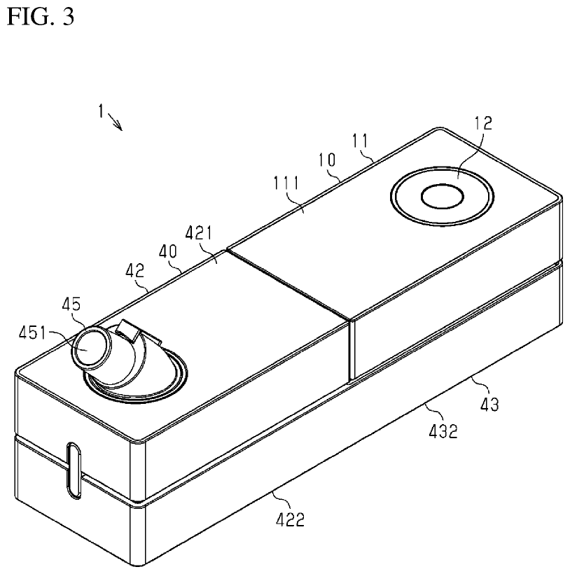

As depicted in FIG. 3, in the state in which the base unit 40 is attached to the main unit 10, the main unit 10 is arranged so as to be separated at a predetermined distance (for example, on the order of 1.5 mm) from the upper surface 431 of the mount part 43 of the base unit 40. With this, the third lead-in part 47 (refer to FIG. 1 and FIG. 2) provided on the base unit 40 communicates with a space outside the CPAP device 1 via a gap between the base unit 40 and the main unit 10. Therefore, without limitation of direction, air intake can be efficiently performed. Also, with the main unit 10 positioned so as to be opposed to the third lead-in part 47, the leakage of the noise from the third lead-in part 47 to the outside is suppressed.

As described above, according to the first embodiment, the following effects can be achieved.

(1-1) Regarding the CPAP device 1, at home, with the base unit 40 attached to the main unit 10, the CPAP device 1 can be used in the above-described first use state. In this first use state, with the base unit 40 attached to the main unit 10, humidified air can be supplied to the user as the CPAP device 1 including the humidifier 51. At sleep away from home or the like, the CPAP device 1 can be used in the second use state in which the base unit 40 is not attached to the main unit 10. In this second use state, since the CPAP device 1 is only with the main unit 10, it is only required to carry only the main unit 10 and high portability is obtained.

(1-2) The first lead-out part 14 includes the base part 142 and the connection cylinder part 143. The base part 142 is connected to the first connection surface 113. The connection cylinder part 143 extends from the base part 142 toward a direction opposite to the first connection surface 113. The base part 142 is formed in a cylindrical shape. The outer peripheral surface of the base part 142 is a cylindrical surface, and the outer shape dimension of the base part 142 is uniform toward a direction protruding from a first connection surface 113 side. The connection cylinder part 143 is formed in a tapered shape, with its outer diameter dimension decreased toward its tip. The second lead-in part 44 has the accommodating part 442 and the connection convex part 444. The accommodating part 442 is recessed from the second connection surface 423 of the second housing 41 toward the inside of the second housing 41. In the accommodating part 442, the first lead-out part 14 of the first housing 11 is accommodated.

According to this configuration, when the first lead-out part 14 is inserted into the second lead-in part 44, the connection cylinder part 143 having the tapered peripheral surface makes the first lead-out part 14 move toward the center of the second lead-in part 44. With this movement of the first lead-out part 14 with respect to the second lead-in part 44, the first lead-out part 14 is guided to a position where the connection convex part 444 of the second lead-in part 44 can be inserted into the connection concave part 144 of the first lead-out part 14. Therefore, the first lead-out part 14 and the second lead-in part 44 can be easily connected.

In addition, with the movement of the first lead-out part 14 with respect to the second lead-in part 44, the first housing 11 and the second housing 41 are relatively guided to a position where the first electrical connection part 15 of the first housing 11 and the second electrical connection part 46 of the second housing 41 are electrically connectable to each other. That is, the first lead-out part 14 and the second lead-in part 44 function as a first guide part and a second guide part which guide the first electrical connection part 15 and the second electrical connection part 46 to a position so that they are electrically connectable to each other. This configuration allows the first electrical connection part 15 and the second electrical connection part 46 to be easily connected.

(1-3) In the CPAP device 1 of the present embodiment, the outer diameter dimension of the connection cylinder part 143 on a base part 142 side is equal to the outer diameter dimension of the base part 142. The outer shape dimension of that base part 142 is smaller than the inner diameter dimension of the accommodating part 442 of the second housing 41. Therefore, as depicted in FIG. 4A, the base part 142 is inserted into the accommodating part 442. The gap (clearance) GA occurs between the outer peripheral surface of the base part 142 and the inner peripheral surface of the accommodating part 442. With this gap GA provided, the first housing 11 and the second housing 41 can be relatively moved along the first connection surface 113 and the second connection surface 423. This movement allows further alignment between the first electrical connection part 15 and the second electrical connection part 46.

(1-4) The outer peripheral surface of the connection convex part 444 is provided with the sealing member 445. The sealing member 445 is accommodated in the groove part provided along the outer peripheral surface of the connection convex part 444. The sealing member 445 is in close contact with the inner peripheral surface of the connection concave part 144 of the first lead-out part 14 of the first housing 11. The sealing member 445 is, for example, an annular seal member. This sealing member 445 seals a portion between the connection convex part 444 and the connection concave part 144. That is, this sealing member 445 can prevent the leakage of the air from the portion between the first lead-out part 14 and the second lead-in part 44. Also, with the sealing member 445 provided on the connection convex part 444 in the accommodating part 442 in the base unit 40, the damage and loss of the sealing member 445 when the main unit 10 is carried can be prevented.

(1-5) In the first use state in which the base unit 40 is attached to the main unit 10, the main unit 10 is arranged so as to be separated at a predetermined distance (for example, on the order of 1.5 mm) from the upper surface 431 of the mount part 43 of the base unit 40. With this, the third lead-in part 47 (refer to FIG. 1 and FIG. 2) provided on the base unit 40 communicates with a space outside the CPAP device 1 via a gap between the base unit 40 and the main unit 10. Therefore, without limitation of direction, air intake can be efficiently performed. Also, with the main unit 10 positioned so as to be opposed to the third lead-in part 47, the leakage of the noise from the third lead-in part 47 to the outside can be suppressed.

In the following, a second embodiment is described. Note that in the following description of the second embodiment, the description of the portions similar to those in the first embodiment is simplified or omitted.

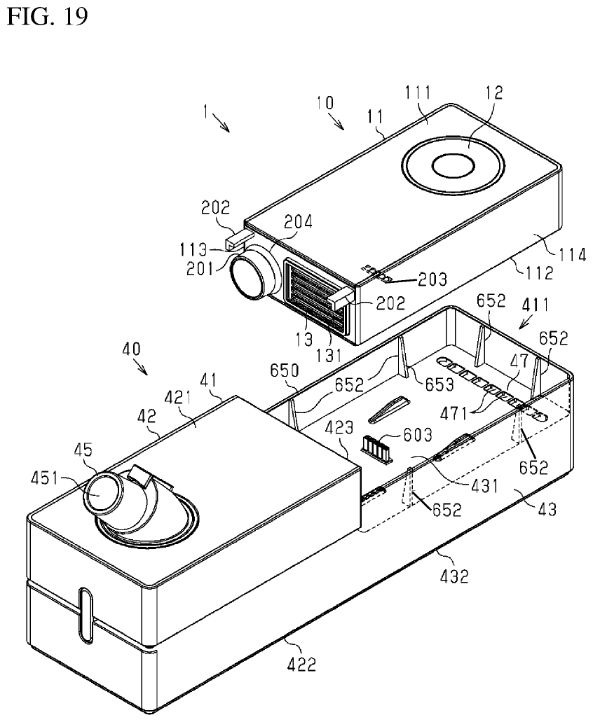

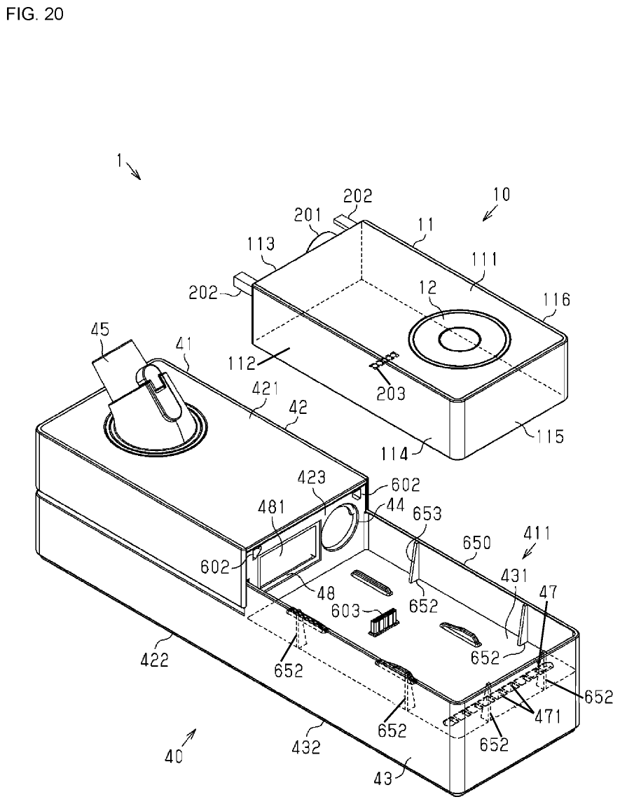

As depicted in FIG. 19 and FIG. 20, the lower surface 112 of the first housing 11 is a third connection surface 112 to be mounted on the base unit 40 in the first use state and mounted on a floor surface, a table, or the like in the second use state. Note that the third connection surface 112 is orthogonal and adjacent to the first connection surface 113.

The first connection surface 113 of the first housing 11 is provided with the first lead-in part 13 which leads air outside the first housing 11 into the first housing 11. The first lead-in part 13 has a plurality of through holes.

Also, on the first connection surface 113, aligned with the first lead-in part 13, a first lead-out part 201 which leads out air from the inside to the outside of the first housing 11 is provided. The first lead-out part 201 protrudes from the first connection surface 113. The first lead-out part 201 is in a cylindrical shape. On the outer peripheral surface of the first lead-out part 201, a portion in contact with the first connection surface 113 has a recessed groove. In that groove, an annular sealing member 204 fits.

Also, from the first connection surface 113 of the first housing 11, two first guide parts 202 protrude. The first guide parts 202 are positioned above the first lead-out part 201 and the first lead-in part 13. Also, the first guide parts 202 are provided at both end portions of the first connection surface 113 in a width direction orthogonal to a vertical direction when the first connection surface 113 is viewed in plan view. That is, each first guide part 202 is provided at an upper corner portion of the first connection surface 113. Also, the first guide part 202 is in a substantially L shape so as to be along the shape of the corner portion when viewed in plan view from a direction orthogonal to the first connection surface 113. Note that the length of the first guide part 202 protruding from the first connection surface 113 is substantially equal to the length of the first lead-out part 201 protruding from the first connection surface 113.

As depicted in FIG. 20, at a substantially center of the third connection surface 112 of the first housing 11, a first electrical connection part 203 is provided for electrically connecting the main unit 10 and the base unit 40. The first electrical connection part 203 is a female connector, and is provided inside the first housing 11 so as not to protrude from the first connection surface 113.

As depicted in FIG. 19 to FIG. 21, the base unit 40 includes the second housing 41. In the second housing 41, the upper surface 431 of the mount part 43 is a fourth connection surface 431 to be opposed to the third connection surface 112 of the first housing 11 in the vertical direction in the first use state.

As depicted in FIG. 20, the second connection surface 423 of the second housing 41 is provided with the second lead-in part 44 for leading air therein from the outside of the second housing 41. The second lead-in part 44 is a through hole penetrating through the inside and outside of the second housing 41. The second connection surface 423 is provided with the third lead-out part 48 for leading air therein from the second housing 41. The third lead-out part 48 is a through hole penetrating through the inside and outside of the second housing 41.

Also, the second connection surface 423 is provided with two second guide parts 602. The second guide parts 602 are through holes penetrating through the inside and outside of the second housing 41. The second guide parts 602 are positioned above the third lead-out part 48 and the second lead-in part 44. Also, the second guide parts 602 are provided at both end portions of the second connection surface 423 in a width direction orthogonal to a vertical direction when the second connection surface 423 is viewed in plan view. That is, each second guide part 602 is provided at an upper corner portion of the second connection surface 423. Also, the second guide part 602 is in a substantially L shape so that the first guide part 202 fits therein, when viewed from a direction orthogonal to the second connection surface 423.

From the fourth connection surface 431, an outer wall 650 protrudes upward. The outer wall 650 extends along three sides of outer edges of the fourth connection surface 431 except a boundary between the second connection surface 423 and the fourth connection surface 431. The protrusion length of the outer wall 650 from the fourth connection surface 431 is, for example, on the order of several millimeters to ten-odd millimeters.

Also, from the fourth connection surface 431, six third guide parts 652 protrude upward. The third guide parts 652 are arranged on the edges of the fourth connection surface 431. Therefore, the third guide parts 652 are connected to the above-described outer wall 650. Two third guide parts 652 are arranged for each of the three sides of the fourth connection surface 431 where the outer wall 650 is present. Therefore, when the fourth connection surface 431 is viewed from above, two third guide parts 652 are present on each of paired long sides of the fourth connection surface 431 opposed in a short direction.

Each third guide part 652 is in a flat plate shape. When viewed in plan view, the third guide part 652 is in a trapezoidal shape with a side on a fourth connection surface 431 side as a long side and an upper side as a short side. A side of the third guide part 652 on a center side of the fourth connection surface 431 is tilted, from a short-side side toward a long-side side, to the center side of the fourth connection surface 431. That is, an edge portion of the third guide part 652 on the center side of the fourth connection surface 431 is a tilted part 653 which is oriented to a first housing 11 side in the first use state and is tilted, from its tip toward its base end, to the center side of the fourth connection surface 431.

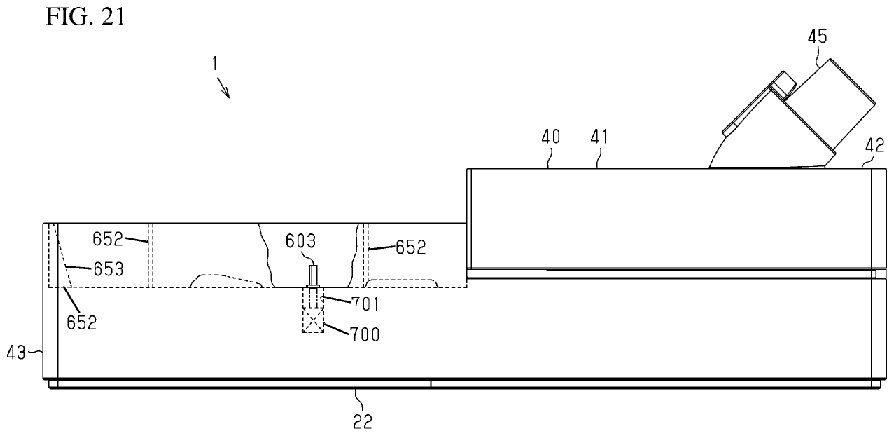

As depicted in FIG. 21, at a substantially center of the fourth connection surface 431 in the second housing 41, a hole part 701 is recessed. Inside the hole part 701, a second electrical connection part 603 is arranged. The second electrical connection part 603 is in contact with the first electrical connection part 203 in the first use state. In the present embodiment, the second electrical connection part 603 is a male connector having a terminal to be attached to and detached from the first electrical connection part 203.

At the bottom part of the hole part 701, a spring 700 as an elastic member is accommodated. The spring 700 is interposed between the bottom surface of the hole part 701 and the second electrical connection part 603. Note in FIG. 21 that the shape of the spring 700 is simplified. The spring 700 presses the second electrical connection part 603 upward. In a state in which any other force does not act on the second electrical connection part 603, the second electrical connection part 603 is pressed by the spring 700 to protrude upward from the fourth connection surface 431. In this manner, the second electrical connection part 603 is movable with respect to a direction orthogonal to the fourth connection surface 431. With the base unit 40 attached to the main unit 10, the second electrical connection part 603 is stored inside the second housing 41. Then, the first electrical connection part 203 and the second electrical connection part 603 are electrically connected.

The first electrical connection part 203 and the second electrical connection part 603 are to perform electrical attachment and detachment between the main unit 10 and the base unit 40. In the present embodiment, the first electrical connection part 203 and the second electrical connection part 603 are used for power supply to the base unit 40 and data transmission and reception using digital signals. Note that power supply and data transmission and reception can also be performed by separate connection parts. For example, noncontact power supply, short-distance wireless communication, and so forth can be used in combination.

Also, as depicted in FIG. 20, the third connection surface 112 and the fourth connection surface 431 are each in a substantially rectangular shape, but the third connection surface 112 has each dimension smaller than that of the fourth connection surface 431. Specifically, the dimension of the third connection surface 112 in the longitudinal direction is shorter than the fourth connection surface 431 by one long side of the third guide part 652. Also, the width dimension of the third connection surface 112 in the width direction is shorter than the fourth connection surface 431 by two long sides of the third guide part 652.

Next, the operation of the second embodiment is described.

When the base unit 40 is attached to the main unit 10, the base unit 40 is first caused to approach from diagonally above as being tilted, the first guide parts 202 on a tip side are partially inserted into the inside of the second guide parts 602. Then, with the tip parts of the first guide parts 202 inserted into the second guide parts 602 as fulcrums, the first housing 11 is rotated so that the third connection surface 112 of the first housing 11 and the fourth connection surface 431 of the second housing 41 are caused to approach. When the first housing 11 is rotated, the first lead-out part 201 protruding from the first connection surface 113 becomes accommodated into the inside of the second lead-in part 44 penetrating through the second connection surface 423. Then, when the first housing 11 is further rotated, the first guide parts 202 are also gradually inserted into the inside of the second guide parts 602 and, finally, substantially the entire first guide parts 202 are inserted into the inside of the second guide parts 602.

Then, when the first housing 11 is rotated as described above and the first guide parts 202 are fully inserted into the second guide parts 602, the first lead-out part 201 is connected to the second lead-in part 44 without a gap by the sealing member 204 provided on the outer periphery of the first lead-out part 201. Also, at the same time when the first lead-out part 201 and the second lead-in part 44 are connected, the first lead-in part 13 and the third lead-out part 48 are connected to each other.

If the first housing 11 is rotated in a state in which the first housing 11 is deviated from the second housing 41 in the width direction, the third connection surface 112 makes contact with the third guide part 652 on either one side arranged in the width direction of the fourth connection surface 431. When the first housing 11 is rotated in this state, the third connection surface 112 approaches the fourth connection surface 431 as sliding on the tilted part 653 of that third guide part 652 to be guided toward the center side of the fourth connection surface 431.

When the third connection surface 112 and the fourth connection surface 431 come close, two third guide parts 652 arranged on the fourth connection surface 431 in the longitudinal direction and the third connection surface 112 may become in contact. In this case, by the tilted parts 653 of these third guide parts 652, the first housing 11 is guided to a direction in which the first connection surface 113 approaches the second connection surface 423.

Also, when the third connection surface 112 and the fourth connection surface 431 come close, the second electrical connection part 603 is pressed by the third connection surface 112 to move so as to be stored inside the second housing 41. That is, when the rotation of the first housing 11 causes the first housing 11 to press the second electrical connection part 603, the spring 700 included inside the second housing 41 contracts to cause the second electrical connection part 603 to move downward.

Then, when the first guide parts 202 and the second guide parts 602 are connected, the state is such that the third connection surface 112 and the fourth connection surface 431 are most close to each other, the second electrical connection part 603 is in contact with the first electrical connection part 203, and the first electrical connection part 203 and the second electrical connection part 603 are electrically connected.

Next, effects of the second embodiment are described.

(2-1) When the base unit 40 is attached to the main unit 10, with the first guide parts 202 and the second guide parts 602 as fulcrums, the first housing 11 is rotated for attachment. Here, it is important to rotate the first housing 11 while each member of the first housing 11 and each member of the second housing 41 are aligned so as to be connected to each other. For example, preferably, the first lead-out part 201 and the second lead-in part 44 are attached without being diagonally deviated so that the first lead-out part 201 is connected to the second lead-in part 44 without a gap. Also, for the connection between the first electrical connection part 203 and the second electrical connection part 603, their terminals are required to be appropriately contacted with each other so as to be electrically connectable.

In the present embodiment, the first lead-out part 201 is provided on the first connection surface 113, and the first electrical connection part 203 is provided on the third connection surface 112. Thus, the operations of the connection of the first lead-out part 201 and the connection of the first electrical connection part 203 can be performed at separate timings. Therefore, the base unit 40 can be easily attached to the main unit 10.

(2-2) The first guide parts 202 are arranged on the first connection surface 113 above the first lead-out part 201 and the first lead-in part 13. Similarly, the second guide parts 602 are arranged on the second connection surface 423 above the third lead-out part 48 and the second lead-in part 44. Therefore, when the base unit 40 is attached to the main unit 10 from diagonally above, the first connection surface 113 and the second connection surface 423 are caused to approach, and the tips of the first guide parts 202 are easily inserted into the second guide parts 602.

(2-3) The first guide parts 202 are provided on the first connection surface 113 at both end portions of the first connection surface 113 in the width direction. Similarly, the second guide parts 602 are provided in the second connection surface 423 at both end portions of the second connection surface 423 in the width direction. In this manner, with two first guide parts 202 and two second guide parts 602 arranged so as to be spaced at distance in the width direction as much as possible, it is possible to suppress tilting of the first housing 11 to the width direction in the course of rotating the first housing 11 with the tips of the first guide parts 202 as fulcrums,

(2-4) In the present embodiment, the second electrical connection part 603 is movable with respect to the fourth connection surface 431 to a direction orthogonal to that fourth connection surface 431. Thus, it is possible to suppress the occurrence of the damages or the like on the second electrical connection part 603 due to a shock when the base unit 40 is attached to the main unit 10.

(2-5) In the present embodiment, the spring 700 is arranged below the second electrical connection part 603. The spring 700 presses the second electrical connection part 603 so that it protrudes above the fourth connection surface 431. For example, when the first housing 11 is removed in the first use state, the second electrical connection part 603 is pressed upward by the spring 700 and is thus automatically returned to the original position as protruding. In this manner, with the second electrical connection part 603 pressed upward, the second electrical connection part 603 is pressed onto the first electrical connection part 203 in the first use state, and the electrical connection therebetween can be ensured.

(2-6) In the present embodiment, the third guide parts 652 each have the tilted part 653 which is oriented to a third connection surface 112 side in the first use state and is tilted, from its protrusion tip toward its base end, to the center side of the fourth connection surface 431. With the presence of the third guide parts 652, when the first housing 11 is attached in a state in which the third connection surface 112 is deviated in the width direction of the fourth connection surface 431, the first housing 11 is guided by the tilted parts 653 of the third guide parts 652 toward the tilting direction. Therefore, the first housing 11 can be attached to the second housing 41 at a correct position.

(2-7) In the present embodiment, two third guide parts 652 are arranged on each of the paired opposed sides of three sides of the fourth connection surface 431 except the boundary of the second connection surface 423. As in the present embodiment, if at least one third guide part 652 is arranged on each of the paired sides, when the base unit 40 is attached to the main unit 10, even if the third connection surface 112 is deviated from the fourth connection surface 431 on either side in the width direction, the third guide parts 652 can guide the first housing 11 to the correct position.

(2-8) In the present embodiment, the third guide parts 652 are arranged on the edges of the fourth connection surface 431, and the third guide parts 652 are connected to the outer wall 650. In this state, the third guide parts 652 and the outer wall 650 are reinforced with each other, and thus the third guide parts 652 and the outer wall 650 are resistant to damage even if external forces are applied.

### (Modifications)

The present embodiments can be implemented by changing as follows.

The present embodiments and the following modifications can be implemented mutually in combination in a technically non-contradictive scope.

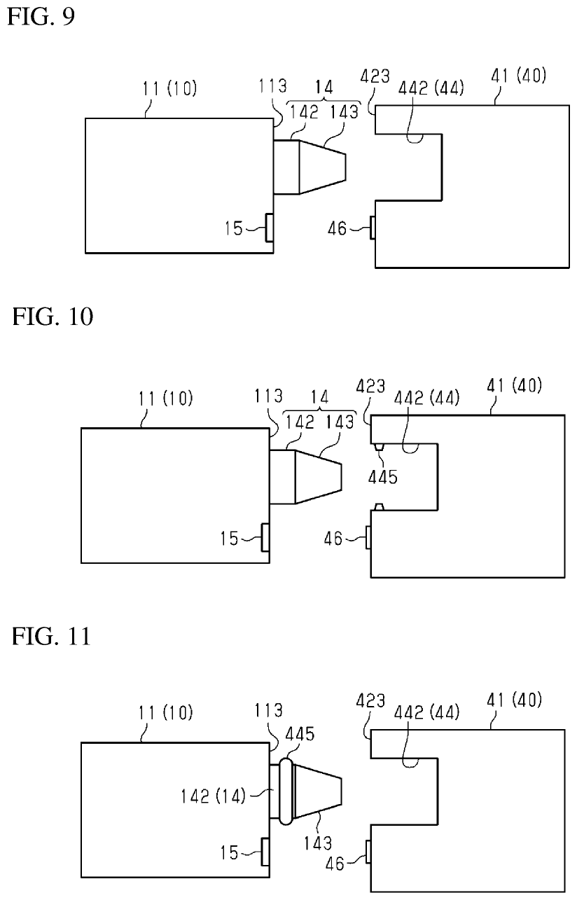

In the first embodiment, as depicted in FIG. 9, the accommodating part 442 of the second lead-in part 44 can be made as a second lead-in port. In this case, with the first lead-out part 14 inserted into the second lead-in port, the first electrical connection part 15 and the second electrical connection part 46 can be easily connected as being aligned.

In the first embodiment, as depicted in FIG. 10, one sealing member 445 may be provided in a cavity portion of the accommodating part 442 as a second lead-in port. This sealing member 445 seals a space between the accommodating part 442 and the base part 142 of the first lead-out part 14. Furthermore, when the first lead-out part 14 is accommodated in the accommodating part 442, the sealing member 445 abuts on the connection cylinder part 143 of the first lead-out part 14, thereby allowing the reduction in the positional deviation of the first lead-out part 14.

In the first embodiment, as depicted in FIG. 11, one sealing member 445 may be provided on the base part 142 of the first lead-out part 14.

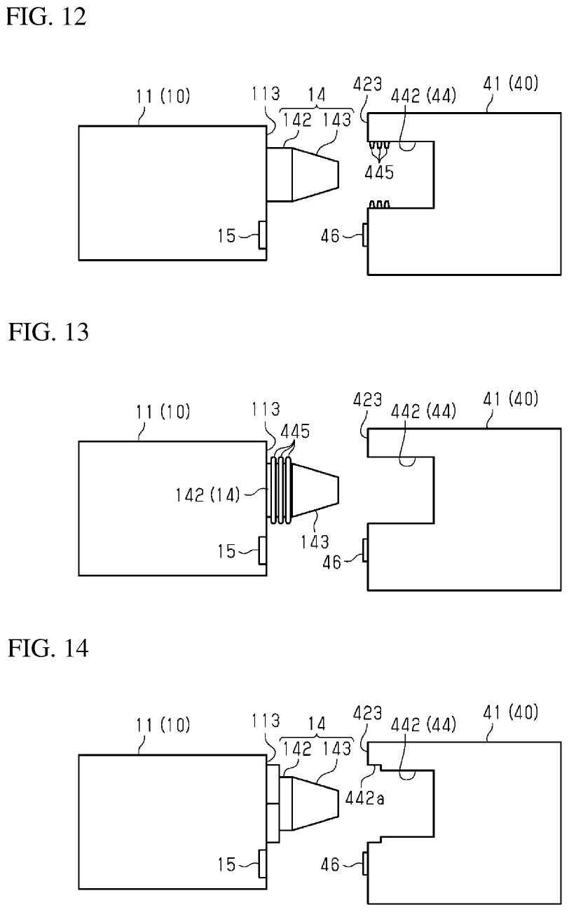

In the first embodiment, as depicted in FIG. 12, three sealing members 445 may be provided in a cavity portion of the accommodating part 442 as a second lead-in port. Also, as depicted in FIG. 13, three sealing members 445 may be provided on the base part 142 of the first lead-out part 14. The number of sealing members 445 can be two or four or more. This allows improvement in sealing performance.

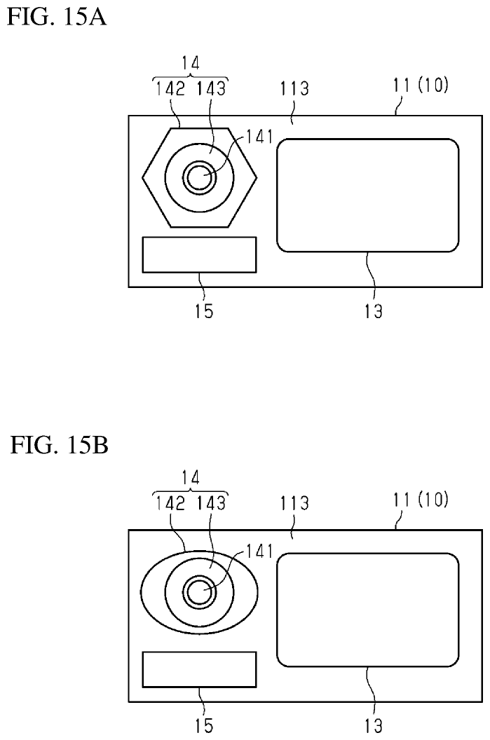

In the first embodiment, as depicted in FIG. 14, the entire or part of the outer peripheral surface of the base part 142 may be made in a non-circular shape when viewed from a tip side of the first lead-out part 14, and a cavity portion 442a opposed to the outer peripheral surface of the base part 142 in the accommodating part 442 as a second lead-in port may be made in a non-circular shape which is substantially similar to that of the portion in the non-circular shape of the first lead-out part 14. For example, as depicted in FIG. 15A, the base part 142 is made in a hexagonal shape. Also, as depicted in FIG. 15B, the base part 142 is made in an oval shape. Note that as a non-circular shape, any shape can be taken, such as a polygon more than a triangle, an oval shape, or a shape with part of the sides of a polygon made in an arc shape. This configuration allows the suppression of the relative rotation between the first lead-out part 14 and the second lead-in part 44 for alignment.

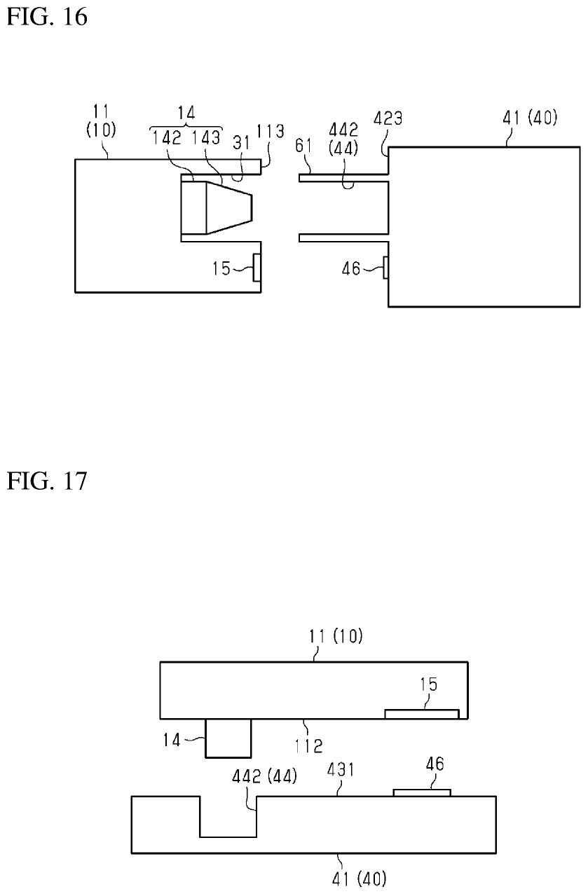

In the first embodiment, as depicted in FIG. 16, it may be configured so that a concave part 31 toward the inside of the first housing 11 is provided in the first connection surface 113 of the main unit 10 and the first lead-out part 14 is disposed in the concave part 31 and a cylinder part 61 is provided on the second connection surface 423 of the base unit 40 and the inside of the cylinder part 61 is taken as the accommodating part 442. According to this configuration, the first lead-out part 14 does not protrude from the first housing 11 in the carried main unit 10, and the damages on the main unit 10 can thus be suppressed.

In the first embodiment, as depicted in FIG. 17, the second lead-in part 44 and the second electrical connection part 46 may be provided on the upper surface 431 of the mount part 43 of the base unit 40, and the first lead-out part 14 and the first electrical connection part 15 may be provided on the lower surface 112 of the main unit 10.

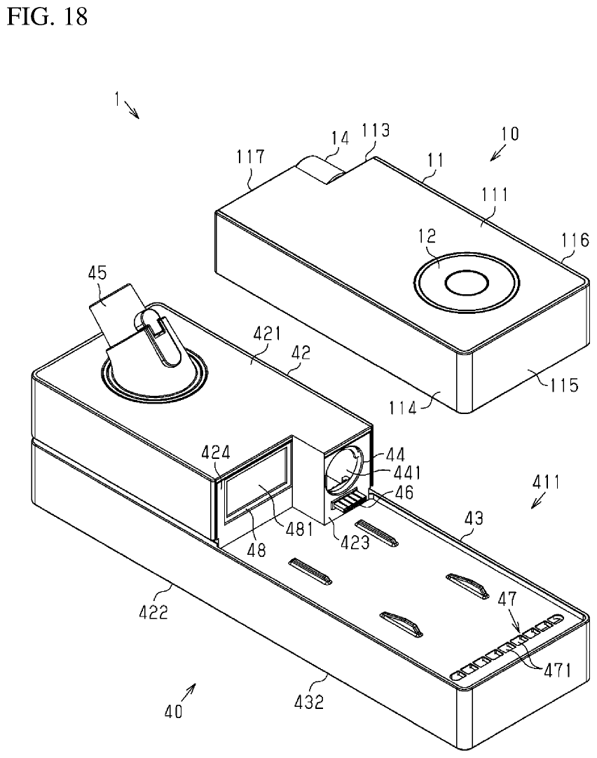

In the first embodiment, as depicted in FIG. 18, the first housing 11 of the main unit 10 has the first connection surface 113 and a third connection surface 117 at a position different from the first connection surface 113 in the longitudinal direction of the main unit 10, and the first lead-in part 13 depicted in FIG. 1 is provided on the third connection surface 117. The second housing 41 of the base unit 40 has a fourth connection surface 424 opposed to the third connection surface 117, and the third lead-out part 48 is provided on the fourth connection surface 424. Also, in the CPAP device 1 configured to have this structure, effects similar to those of the above-described embodiment can be achieved. Note that the first connection surface 113 and the third connection surface 117 are preferably substantially parallel and the second connection surface 423 and the fourth connection surface 424 are preferably substantially parallel.

In the first embodiment, in the main unit 10, the first lead-out part 14 and the first electrical connection part 15, and the second lead-in part 44 and the second electrical connection part 46 may be provided on different surfaces. For example, the first electrical connection part 15 and the second electrical connection part 46 may be provided on the lower surface 112 of the main unit 10 and the upper surface 431 of the mount part 43 of the base unit 40, respectively, and the first lead-out part 14 and the second lead-in part 44 may be provided on the first connection surface 113 of the first housing 11 and the second connection surface 423 of the second lead-in part 44, respectively. Also, in the first housing 11 and the second housing 41 configured as depicted in FIG. 18, the first electrical connection part 15 and the second electrical connection part 46 may be provided on the third connection surface 117 and the fourth connection surface 424.

In the second embodiment, the number of sealing member 204 does not matter. The sealing members 204 may be increased or omitted unless the connection between the first lead-out part 201 and the second lead-in part 44 is hindered.

In the second embodiment, the arrangement of the sealing member 204 is not limited to the outer periphery of the first lead-out part 201. For example, it may be provided on the inner peripheral surface of the second lead-in part 44.

In the second embodiment, only one first guide part 202 or three or more first guide parts 202 may be provided on the first connection surface 113. When the number of first guide parts 202 is changed, the corresponding numbers of second guide parts 602 are provided in the second connection surface 423 so as to match the number of first guide parts 202. Also, in that case, each second guide part 602 is arranged at a position capable of fitting in the first guide part 202 in the first use state.

In the second embodiment, the shape of the first guide part 202 is not limited to an L shape. For example, it may be a columnar protrusion. Note that when the shape of the first guide part 202 is changed, it is only required that the shape of the second guide part 602 is changed to a shape capable of fitting in the first guide part 202.

In the second embodiment, the length of the first guide part 202 protruding from the first connection surface 113 is only required to be longer than or equal to the length of the first lead-out part 201 protruding from the first connection surface 113. However, the length of the first guide part 202 is preferably a length which allows quick rotation of the first housing 11 when the base unit 40 is attached to the main unit 10.

In the second embodiment, the second guide part 602 may be a member protruding in an L shape along the shape of the corner portion when viewed in plan view from a direction orthogonal to the second connection surface 423, and the first guide part 202 may be an L-shaped through hole when viewed from a direction orthogonal to the first connection surface 113. In this manner, it is only required that a convex part is formed on either one of the first connection surface 113 and the second connection surface 423 and a concave part is formed on the other so that the first guide part 202 and the second guide part 602 fit in each other.

In the second embodiment, the second guide part 602 may not be a through hole as long as it has a shape recessed inward from the outer side portion of the second connection surface 423, and may be a closed-end hole.

In the second embodiment, the first electrical connection part 203 may be arranged on any portion of the third connection surface 112. However, the first electrical connection part 203 is preferably arranged at a position not interfering with the third lead-in part 47 arranged on the third connection surface 112. Also, it is only required that the position of the second electrical connection part 603 on the fourth connection surface 431 is changed so as to be aligned with the position of the first electrical connection part 203.

In the second embodiment, regarding the third guide part 652, if the tilted part 653 is provided which is oriented to a first housing 11 side in the first use state and is tilted, from its tip toward its base end, to the center side of the fourth connection surface 431, the shape of the third guide part 652 may be changed as appropriate. For example, it may be a columnar shape or a shape spreading over the entire outer wall 650.

In the second embodiment, as for the arrangement of the third guide parts 652, one may be arranged on each of the paired opposed sides of three sides of the fourth connection surface 431 except the boundary of the second connection surface 423. Also, the number of third guide parts 652 is not limited to that in the second embodiment. For example, no third guide part 652 may be arranged on a not-opposed side of three sides of the fourth connection surface 431 except the boundary of the second connection surface 423. In that case, when the base unit 40 is attached to the main unit 10, some way is preferably taken at the time of the attachment, such as holding the first housing 11 by hand so as to prevent the occurrence of a positional deviation with respect to the longitudinal direction.

In the second embodiment, the length of the protrusion of the outer wall 650 may be changed as appropriate. Also, the outer wall 650 may be disposed on at least a part of the edges of the third connection surface 112.

In the second embodiment, the outer wall 650 can be omitted.

In the second embodiment, a member pressing the second electrical connection part 603 may not be the spring 700. For example, the second electrical connection part 603 may be pressed with a structure in which the second electrical connection part 603 is supported by an elastic member such as a columnar rubber and, when the second electrical connection part 603 is pressed, the columnar elastic member elastically contracts to cause the second electrical connection part 603 to be stored inside the second housing 41.

In the second embodiment, the structure of allowing the second electrical connection part 603 to be movable is not limited to an elastic member. For example, a lever or the like mechanically coupled to the second electrical connection part 603 may be provided on the second housing 41 to cause the second electrical connection part 603 to be movable by operating this lever.

In each embodiment, the first control part 21 of the main unit 10 may be configured to control the humidifier 51 of the base unit 40, and the second control part 53 of the base unit 40 can be omitted. The first control part 21 detects a heater temperature by the temperature sensor 513 by using the first electrical connection part 15 and the second electrical connection part 46 to control the heater 512 based on the heater temperature.
1 CPAP device
2 air tube
3 mask
10 main unit (first unit)
13 first lead-in part
14 first lead-out part
21 first control part
22 air blower
40 base unit (second unit)
44 second lead-in part
45 second lead-out part
51 humidifier
53 second control part

1. A CPAP device for feeding air led inside the device to an airway of a user, the CPAP device comprising:
a first unit; and
a second unit attachable to or detachable from the first unit, wherein
the first unit includes a first housing having a first lead-in part and a first lead-out part, an air blower for leading out air led in from the first lead-in part from the first lead-out part, a first electrical connection part provided on the first housing, and a first guide part,
the second unit includes a second housing having a second lead-in part and a second lead-out part, a humidifier which humidifies air led therein from the second lead-in part, a second electrical connection part electrically connectable to the first electrical connection part, and a second guide part, and
the first guide part and the second guide part are configured to guide, with a connection between the first lead-out part and the second lead-in part, the first electrical connection part and the second electrical connection part to a position where the first electrical connection part and the second electrical connection part are electrically connectable to each other.

2. The CPAP device according to claim 1, wherein
the first housing has a first connection surface provided with the first guide part,
the second housing has a second connection surface opposed to the first connection surface in a first use state in which the second unit is attached to the first unit and is provided with the second guide part,
the first guide part is a convex part with a tip protruding from the first connection surface, and
the second guide part is a concave part recessed from the second connection surface and into which the convex part is inserted.

3. The CPAP device according to claim 1, wherein
the first housing has a first connection surface provided with the first guide part,
the second housing has a second connection surface opposed to the first connection surface in a first use state in which the second unit is attached to the first unit and is provided with the second guide part,
the second guide part is a convex part with a tip protruding from the second connection surface, and
the first guide part is a concave part recessed from the first connection surface and into which the convex part is inserted.

4. The CPAP device according to claim 2, wherein
the first electrical connection part is provided on the first connection surface, and
the second electrical connection part is provided on the second connection surface.

5. The CPAP device according to claim 2, wherein
the convex part includes an annular base part and a connection cylinder part extending from the base part,
the base part has an outer diameter uniform toward the tip of the convex part, and
a gap is provided between an outer peripheral surface of the base part and an inner surface of the concave part.

6. The CPAP device according to claim 5, wherein
the connection cylinder part is in a tapered shape, with an outer diameter decreased toward the tip of the convex part.

7. The CPAP device according to claim 5, wherein
at least either one of the outer peripheral surface of the base part and the inner surface of the concave part opposed to the outer peripheral surface has at least one sealing member interposed between the base part and the concave part.

8. The CPAP device according to claim 5, wherein
the outer peripheral surface of the base part is in a non-circular shape when viewed from a tip side of the convex part, and
a portion of an inner peripheral surface of the concave part opposed to the outer peripheral surface of the base part is in a non-circular shape similar to the non-circular shape of the outer peripheral surface when viewed from a cavity side of the concave part.

9. The CPAP device according to claim 2, wherein
the first housing has a third connection surface provided with the first lead-in part, and
the second housing has a fourth connection surface opposed to the third connection surface in the first use state in which the second unit is attached to the first unit, and a third lead-out part provided on the fourth connection surface.

10. The CPAP device according to claim 1, wherein
the first unit includes a first control part for controlling the air blower,
the second unit includes a second control part for controlling the humidifier, and
the first control part is configured to transmit a digital signal to the second control part, the second control part is configured to transmit a digital signal to the first control part, or the first control part and the second control part are mutually configured to transmit digital signals.

11. The CPAP device according to claim 1, wherein
the first unit includes a control part for controlling the air blower,
the control part is connected to the humidifier via the first electrical connection part and the second electrical connection part to control the humidifier.

12. The CPAP device according to claim 2, wherein
the first housing has a third connection surface adjacent to the first connection surface and opposed to the second housing in the first use state,
the second housing has a fourth connection surface adjacent to the second connection surface and opposed to the third connection surface in the first use state,
the first lead-out part is provided on the first connection surface,
the second lead-in part is provided on the second connection surface,
the first electrical connection part is provided on the third connection surface, and
the second electrical connection part is provided on the fourth connection surface.

13. The CPAP device according to claim 12, wherein
in a direction in which the third connection surface and the fourth connection surface are opposed to each other, when a side of the third connection surface is taken as being above and a side of the fourth connection surface is taken as being below,
the first guide part is positioned on the first connection surface above the first lead-out part, and
the second guide part is positioned on the second connection surface above the second lead-in part.

14. The CPAP device according to claim 12, wherein
the first guide part includes two first guide parts provided at both end portions of the first connection surface in a width direction, and
the second guide part includes two second guide parts provided at both end portions of the second connection surface in a width direction so as to be opposed to the pair of first guide parts.

15. The CPAP device according to claim 12, wherein
the second electrical connection part is movable with respect to the fourth connection surface to a direction orthogonal to the fourth connection surface.

16. The CPAP device according to claim 15, wherein
the second housing is provided with an elastic member for pressing the second electrical connection part to a side of the first electrical connection part in the first use state in which the second unit is attached to the first unit.

17. The CPAP device according to claim 15, wherein
the fourth connection surface is provided with a third guide part protruding from an edge of the fourth connection surface, and
the third guide part has a tilted part oriented to a side of the third connection surface in the first use state in which the second unit is attached to the first unit and tilted, from a protruding tip toward a base end, to a center side of the fourth connection surface.

18. The CPAP device according to claim 17, wherein
the third guide part includes one or more third guide parts arranged on each of paired opposed sides of three sides of the fourth connection surface except a boundary of the second connection surface.

19. The CPAP device according to claim 3, wherein
the first electrical connection part is provided on the first connection surface, and
the second electrical connection part is provided on the second connection surface.

20. The CPAP device according to claim 3, wherein
the convex part includes an annular base part and a connection cylinder part extending from the base part,
the base part has an outer diameter uniform toward the tip of the convex part, and
a gap is provided between an outer peripheral surface of the base part and an inner surface of the concave part.
